(19) United States
(12) Patent Application Publication
(10) Pub. No.: US 2013/0309131 A1
(43) Pub. Date: Nov. 21, 2013

(54) Dynamic Ultraviolet Lamp Ballast System

(71) Applicant: Hayward Industries, Inc., Elizabeth, NJ (US)

(72) Inventor: Rolf Engelhard, Prescott, AZ (US)

(21) Appl. No.: 13/897,623

(22) Filed: May 20, 2013

Related U.S. Application Data
(60) Provisional application No. 61/649,888, filed on May 21, 2012.

(51) Int. Cl. H05B 41/36; A61L 2/10

(52) U.S. Cl. CPC H05B 41/36; A61L 2/10
USPC 422/24; 315/309; 250/436

(57) ABSTRACT

Exemplary embodiments are directed to lamp ballast systems, generally including a lamp, at least one temperature sensor, a ballast and a processor. The at least one temperature sensor can be positioned proximate to the lamp or incorporated into the lamp. The ballast provides an electrical current to the lamp. The processor receives a sensed temperature from the at least one temperature sensor and, in response to the sensed temperature, directs a control signal to the ballast to regulate the electrical current provided to the lamp to maintain the lamp at an optimal operating temperature. Exemplary embodiments are also directed to methods of maintaining a lamp at an optimal operating temperature, generally including providing a lamp ballast system, receiving a sensed temperature, and directing a control signal to the ballast to regulate the electrical current provided to the lamp to maintain the lamp at the optimal operating temperature.

### CROSS-REFERENCE TO RELATED APPLICATIONS

The present application claims the benefit of a co-pending provisional patent application entitled “Dynamic Temperature Compensating UV Lamp Ballast,” which was filed on May 21, 2012, and assigned Ser. No. 61/649,888. The entire content of the foregoing provisional application is incorporated herein by reference.

### TECHNICAL FIELD

The present disclosure relates to lamp ballast systems and associated methods and, in particular, to lamp ballast systems for providing dynamic temperature compensation.

### BACKGROUND

It is known in the swimming pool industry that ultraviolet (UV) germicidal irradiation can be harmful to microorganisms. Ultraviolet light in the 254 nanometer range can effectively destroy the nucleic acids in microorganisms, which disrupts their DNA and removes their reproductive capabilities, thereby killing them. It is also known in the industry that UV light in the 185 nanometer range converts oxygen to ozone.

One effective way to generate ultraviolet light in the 254 nanometer and 185 nanometer ranges is by means of mercury vapor lamps. The most common of these lamps are low pressure, mercury vapor UV lamps. These lamps come in the form of (i) low pressure, low output lamps, (ii) low pressure, standard output lamps, (iii) low pressure, high output lamps, and (iv) low pressure, amalgam lamps.

Typically, the different types of low pressure UV lamps have a UV efficiency of approximately 25% to 40%. Thus, depending on the type of lamp being implemented, between 25% and 40% of the total input energy converts to the germicidal light frequency in the 254 nanometer range. As is known in the industry, the efficiency of low pressure UV lamps can be largely affected by the internal operating temperature of the lamps.

The internal operating temperature of low pressure UV lamps can generally be measured by a “cold-spot” within the lamp, i.e., the coolest section of the lamp. Typically, the ideal cold-spot temperature of the low pressure, low output, standard output, and high output UV lamps is approximately 107° F. The ideal cold-spot temperature of an amalgam UV lamp is typically approximately 160° F. Any temperature variation above or below the ideal operating temperature of the UV lamps can decrease the effective UV output by as much as 1% for each 1.5° F. temperature variation. Thus, a UV lamp that is operated 15° F. above or below its ideal operating temperature will generally experience an approximately 10% decrease of its effective UV output.

In general, the operating temperature of a lamp can be affected by the following factors: (i) the lamp current, which determines the amount of electrical energy the lamp consumes, and (ii) the temperature of the environment surrounding the lamp, which affects the cooling or heating of the lamp. UV lamps are typically installed in an environment that is cooler than the ideal operating temperature of the lamp. The lamp can generally be placed inside a secondary quartz sleeve to reduce the heat loss from the lamp in the cooler environment. This arrangement creates an insulating air space between the lamp surface and the fluid medium, e.g., liquid or gas medium, in which the lamp operates.

UV lamps are typically used to purify a fluid, e.g., air or water. Air purification, for example, can occur in a forced-air heating system of a building where average air temperatures may be approximately 70° F. As a further example, air purification can occur in a commercial freezer where average air temperatures may be −20° F. The UV lamp in the freezer generally requires a higher electrical current to maintain an ideal operating temperature when compared to the UV lamp in the heating system. The supply of higher or lower electrical current to a UV lamp can be achieved by choosing a different lamp ballast for each condition. In particular, a specific ballast can be selected for each respective UV lamp based on the environment surrounding the lamp to appropriately control the supply of electrical current to the lamp.

However, in an application where the environmental temperature changes periodically, e.g., a swimming pool, the substantially linear supply of electrical current to the lamp by the selected ballast generally causes the lamp to operate below or above an ideal operating temperature of the lamp. For example, in a seasonal swimming pool, the water may be heated to a temperature of 85° F. in the summer and the temperature can drop to 50° F. in the winter. As would be understood by those of ordinary skill in the art, in the seasonal swimming pool scenario described above, the electrical current required to maintain the ideal operating temperature of the lamp would need to be higher in the winter than in the summer. However, due to the linear supply of electrical current to the lamp by the ballast, a loss in the UV output is generally incurred when the temperature varies from the ideal operating temperature.

Thus, a need exists for a UV lamp ballast which dynamically compensates a supply of electrical current to maintain an ideal operating temperature of the UV lamp. These and other needs are addressed by the lamp ballast systems and associated methods of the present disclosure.

### SUMMARY

In accordance with embodiments of the present disclosure, exemplary lamp ballast systems are provided, generally including a lamp, e.g., a UV lamp, at least one temperature sensor, a ballast and a processor. The at least one temperature sensor can be positioned proximate to the lamp or incorporated into the lamp. The ballast can provide an electrical current to the lamp. The processor generally receives a sensed temperature, e.g., an environment operating temperature, a current cold-spot temperature, and the like, from the at least one temperature sensor. In response to the sensed temperature, e.g., an environment operating temperature, a lamp cold-spot temperature, and the like, the processor can direct a control signal to the ballast to regulate the electrical current provided to the lamp to maintain the lamp at an optimal operating temperature, e.g., an optimal lamp cold-spot temperature. Thus, as the environment operating temperature and/or the current cold-spot temperature of the lamp changes, the processor can regulate the supply of electrical current to the lamp from the ballast to maintain the lamp at the optimal operating temperature. The optimal lamp cold-spot temperature can, in turn, generate or permit an optimal ultraviolet output intensity to be emitted from the lamp.

The exemplary systems generally include a housing. The housing includes an inlet and an outlet for introducing fluid to be purified into the housing and for discharging purified fluid from the housing, respectively. The lamp can be positioned within the housing. The at least one temperature sensor, e.g., a thermocouple, a thermistor, a microchip, and the like, can be positioned inside the housing at the inlet, at the outlet, or at both the inlet and the outlet. In some embodiments, the at least one temperature sensor can be positioned within the lamp. The processor generally includes a database therein configured to be programmed with at least one algorithm. The at least one algorithm can represent a relationship between the electrical current required to maintain the lamp at the optimal operating temperature and a variety of sensed temperatures.

In accordance with embodiments of the present disclosure, exemplary methods of maintaining a lamp at an optimal operating temperature. The exemplary methods generally include providing a lamp ballast system. The lamp ballast system generally includes a lamp, at least one temperature sensor, a ballast and a processor. The at least one temperature sensor can be positioned proximate to the lamp or incorporated into the lamp. The ballast can provide an electrical current to the lamp. The exemplary methods include receiving a sensed temperature, via the processor, from the at least one temperature sensor. In response to the sensed temperature, the methods include directing a control signal, via the processor, to the ballast to regulate the electrical current provided to the lamp to maintain the lamp at the optimal operating temperature.

The lamp ballast system generally includes a housing. The housing includes an inlet and an outlet for introducing fluid to be purified into the housing and for discharging purified fluid from the housing, respectively. The lamp can be positioned within the housing. The methods include positioning the at least one temperature sensor inside the housing at the inlet, at the outlet, or at both the inlet and the outlet. In some embodiments, the temperature sensor can be positioned inside the lamp. The methods generally include sensing the sensed temperature at one or both of an operating environment surrounding the lamp and/or a lamp cold-spot. Maintaining the lamp at the optimal operating temperature generally includes generating or permitting an optimal ultraviolet output intensity to be emitted from the lamp.

In some embodiments, the methods include starting the lamp by providing a maximum operating current to the lamp. The methods further include directing a reduction control signal to the ballast to reduce the electrical current provided to the lamp when the optimal operating temperature has been reached. In some embodiments, the methods include starting the lamp by providing a minimum operating current to the lamp. The methods further include directing an increase control signal to the ballast to increase the electrical current provided to the lamp to reach the optimal operating temperature. The exemplary methods generally include programming at least one algorithm into a database of the processor. The at least one algorithm can represent a relationship between the electrical current required to maintain the lamp at the optimal operating temperature and a variety of sensed temperatures.

In accordance with embodiments of the present disclosure, exemplary lamp ballast systems to provide an optimal setting for a UV lamp are provided, the systems generally including a temperature sensor, a processor and a ballast. The temperature sensor can be proximate the UV lamp for determining a temperature of an environment near the UV lamp. The processor can receive the temperature from the temperature sensor. The processor can further generate a control signal for controlling a temperature of the UV lamp based on the temperature of the environment near the UV lamp. The ballast can be responsive to the control signal for providing an electrical current to the UV lamp to maintain the temperature of the UV lamp at the optimal setting. Methods of dynamic temperature compensation with a lamp ballast system are also provided.

In accordance with embodiments of the present disclosure, exemplary non-transitory computer readable storage mediums storing instructions are provided. Execution of the instructions by a processor causes the processor to implement a method of maintaining a lamp at an optimal operating temperature, generally including receiving a sensed temperature, via the processor, from at least one temperature sensor of a lamp ballast system. The lamp ballast system generally includes the lamp, the at least one temperature sensor positioned proximate to the lamp or incorporated into the lamp, a ballast providing an electrical current to the lamp, and the processor. In response to the sensed temperature, the method includes directing a control signal, via the processor, to the ballast to regulate the electrical current provided to the lamp to maintain the lamp at the optimal operating temperature.

Other objects and features will become apparent from the following detailed description considered in conjunction with the accompanying drawings. It is to be understood, however, that the drawings are designed as an illustration only and not as a definition of the limits of the invention.

### BRIEF DESCRIPTION OF THE DRAWINGS

To assist those of skill in the art in making and using the disclosed lamp ballast systems and associated methods, reference is made to the accompanying figures, wherein:

FIG. 1 is a block diagram of an exemplary lamp ballast system according to the present disclosure; and

FIG. 2 is a chart illustrating a representative relationship between a supply of electrical current versus a UV lamp output intensity.

### DESCRIPTION OF EXEMPLARY EMBODIMENTS

The exemplary systems described herein are generally directed to mechanisms to be incorporated with a lamp ballast of a UV lamp. Exemplary methods for operating such mechanisms are also provided for automatically adjusting a lamp current to maintain an ideal lamp operating temperature. The exemplary system generally includes a lamp ballast, a temperature sensor, and a processor. The lamp ballast can provide varying electrical currents to the UV lamp. The temperature sensor can measure temperature of an environment around the UV lamp. The processor can monitor a signal from the temperature sensor and can further regulate the ballast according to the monitored temperature to maintain an optimal lamp temperature.

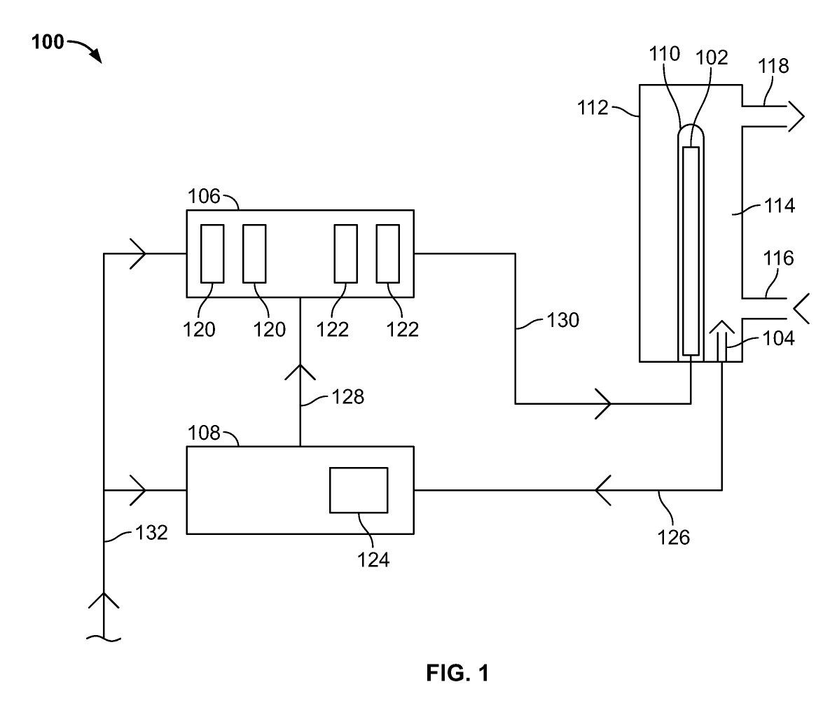

With reference to FIG. 1, a block diagram of an exemplary lamp ballast system 100 is provided. The exemplary system 100 generally includes a lamp 102, e.g., a UV lamp, a temperature sensor 104, a ballast 106, and a processor 108, e.g., a processing device. The lamp 102 can be configured to generate UV light in the 254 nanometer range at a peak UV output intensity to effectively destroy nucleic acids in microorganisms. In some embodiments, the lamp 102 can be positioned inside a sleeve 110, e.g., a quartz sleeve. The lamp 102 and the sleeve 110 can be further positioned within a housing 112. The housing 112 can be configured and dimensioned to receive the lamp 102 and the sleeve 110 therein, and includes an internal space 114 for receiving a fluid, e.g., air, water, and the like, to be purified by the UV light from the lamp 102.

In particular, the housing 112 includes an inlet 116 for receiving a fluid to be purified by the UV light from the lamp 102 into the internal space 114. The housing 112 further includes an outlet 118 for discharging purified fluid out of the internal space 114 into the operating environment, e.g., a swimming pool. Although discussed herein as purifying swimming pool water, it should be understood that the exemplary system 100 and the lamp 102 can be used to purify and disinfect air and other fluids.

The temperature sensor 104 can be, e.g., a thermocouple, a thermistor, a microchip, any other device capable of sensing temperature, and the like. As illustrated in FIG. 1, the temperature sensor 104 can be incorporated into and positioned within the housing 112 proximate to the lamp 102. In particular, FIG. 1 illustrates the temperature sensor 104 positioned in the media or fluid surrounding the lamp 102 in the internal space 114 of the housing 112. Although illustrated as positioned near the inlet 116 of the housing 112, in some embodiments, the temperature sensor 104 can be positioned in the proximity of the inlet 116, in the proximity of the outlet 118, one temperature sensor 104 can be positioned in the proximity of the inlet 116 and a second temperature sensor 104 can be positioned in the proximity of the outlet 118 and an average of the two temperatures can be calculated, the temperature sensor 104 can be positioned in any other location or region within the internal space 114 of the housing 112, and the like.

The temperature sensor 104 can thereby measure the temperature of the fluid in the internal space 114 of the housing 112 at the inlet 116, at the outlet 118, at the inlet 116 and the outlet 118 and calculate an average between the inlet 116 and the outlet 118 temperatures, and at any other location or region within the internal space 114, respectively. The temperature sensor 104 can be further configured to send a signal to the processor 108 indicating the measured or sensed temperature of the fluid in the internal space 114 of the housing 112. By including the temperature sensor 104 in the internal space 114 of the housing 112, the cold-spot temperature of the lamp 102 can be indirectly measured by measuring the temperature of the lamp 102 operating environment. The algorithms and relationships discussed herein can thereby be dependent on the lamp 102 operating environment for maintaining an optimum lamp 102 cold-spot temperature.

In some embodiments (not shown), as an alternative or in addition to the temperature sensor 104 positioned in the internal space 114 of the housing 112, a temperature sensor 104 can be incorporated into the lamp 102 to measure the cold-spot temperature of the lamp 102. The temperature sensor 104 can then send the measured or sensed cold-spot temperature of the lamp 102 to the processor 108. By including a temperature sensor 104 within the lamp 102, the lamp 102 cold-spot temperature can be directly measured. The algorithms and relationships discussed herein can thereby be dependent on the lamp 102 cold-spot temperature for maintaining an optimum lamp 102 cold-spot temperature.

The lamp ballast 106 of the system 100 can be adjustable to provide varying electrical currents through the lamp 102. The ballast 106 includes one or more resistors 120 and one or more capacitors 122 therein configured and arranged to controllably provide electric current to the lamp 102. Although illustrated as including two resistors 120 and two capacitors 122 in FIG. 1, it should be understood that the exemplary ballast 106 can include one or more resistors 120 and/or capacitors 122. A change of a resistance value for a resistor 120 and/or a capacitance value for a capacitor 122 can change the electrical current being provided to and passing through the lamp 102.

The processor 108 of the system 100 generally acts as a controller to monitor temperature signals sent from the temperature sensor 104 and, according to internal programming within the processor 108, to regulate the ballast 106 to provide the ideal current to maintain the lamp 102 in an optimal lamp temperature, e.g., an optimum lamp cold-spot temperature. For example, the processor 108 can maintain the lamp 102 at an optimal lamp temperature by regulating the resistance value for a resistor 120 and/or the capacitance valve of a capacitor 122 in the ballast 106 to change the electrical current being provided to and passing through the lamp 102. In particular, the processor 108 generally includes a programmable database 124 located therein which can be programmed with one or more algorithms including correlation data for electrical current and a variety of operating temperatures and/or cold-spot temperatures. The database 124 can store the algorithms, correlation data and/or instructions related to the algorithms with respect to regulating the current being supplied by the ballast 106. In some embodiments, the instructions can be implemented using non-transitory computer readable medium technologies, such as a floppy drive, a hard drive, a tape drive, solid state storage devices, a flash drive, an optical drive, read only memory (ROM), random access memory (RAM), and the like. In some embodiments, the processor 108 can operate to execute the algorithms or instructions stored in the database 124 and can store data resulting from the executed algorithms or instructions, which may be presented via, for example, a graphical user interface (GUI). For example, the GUI can display the environment operating temperature, the current cold-spot temperature, the optimum cold-spot temperature, the UV output intensity, and the like.

The algorithms generally include a plurality of relationships between an operating environment temperature and/or a cold-spot temperature and the electrical current input required to maintain the lamp 102 at an optimal lamp 102 cold-spot temperature for at least one lamp 102 type. The relationships generally include correlation data between optimum or ideal lamp 102 currents for each specific water temperature to maintain the lamp 102 at an optimum lamp temperature. Thus, from the correlation data in the algorithms, the processor 108 can be programmed to automatically regulate the ballast 106 to feed the ideal current to the lamp 102 for each given water temperature. Different lamp 102 types, i.e., lamps 102 having different optimum cold-spot temperatures, can include processors 108 therein programmed with alternative algorithms based on the relationships between electrical currents and measured temperatures for maintaining the lamp-specific optimum cold-spot temperature.

For example, for an operating environment temperature of approximately 50° F., a current of approximately 800 mA supplied to the lamp 102 can generate an ideal lamp temperature of approximately 117° F. Similarly, for an operating environment temperature of approximately 80° F., the current can be reduced to approximately 500 mA to maintain the ideal lamp temperature of approximately 117° F. These algorithms and values of temperature with respect to current supplied for ideal lamp temperatures can be programmed into the processor 108 and stored in the database 124. As described above, the ideal lamp temperature can vary depending on the type of lamp 102 being implemented. Thus, the algorithms can vary to appropriately reflect the optimal lamp temperatures for the type of lamp 102 being implemented.

By implementing the programmed algorithms, in response to signals received from the temperature sensor 104 through, e.g., an electrical cable 126, the processor 108 can automatically regulate the current provided by the ballast 106 to the lamp 102 via a control loop by providing a variable resistance and/or a variable capacitance, or any other signal required by the ballast 106 to vary the current, to the ballast 106. For example, the processor 108 can send regulatory signals to the ballast 106 through, e.g., an electrical cable 128. Although illustrated as a one-way signal, in some embodiments, the processor 108 can receive signals from the ballast 106 through the electrical cable 128 indicating the regulated current value being supplied to the lamp 102, e.g., a feedback loop. The ballast 106, in turn, can provide or feed a regulated current to the lamp 102 through, e.g., an electrical cable 130. Electrical power for operating the ballast 106 and the processor 108 can be supplied to the ballast 106 and the processor 108 from a power source (not shown) through, e.g., an electrical cable 132. Although discussed herein as electrical cables, in some embodiments, wireless transfer of signals or power between the temperature sensor 104, the ballast 106, the processor 108, and/or the power source can be performed over a wireless network.

As an example, for a lamp 102 having an ideal lamp temperature, i.e., an optimum lamp 102 cold-spot temperature, of 117° F., the processor 108 can include algorithms programmed therein for regulating the current being supplied to the ballast 106 for maintaining the lamp 102 at the ideal lamp temperature of 117° F. Thus, if the operating environment temperature, e.g., swimming pool water in the winter, is approximately 50° F., the processor 108 can regulate the resistance and/or capacitance of the ballast 106 to supply a current of approximately 800 mA to the lamp 102 to maintain the lamp 102 at the ideal lamp temperature of 117° F. If the operating environment temperature changes, e.g., swimming pool water in the summer, to approximately 80° F., the processor 108 can regulate the resistance and/or capacitance of the ballast 106 to reduce the current supplied to the lamp 102 to approximately 500 mA to maintain the lamp 102 at the ideal lamp temperature of 117° F.

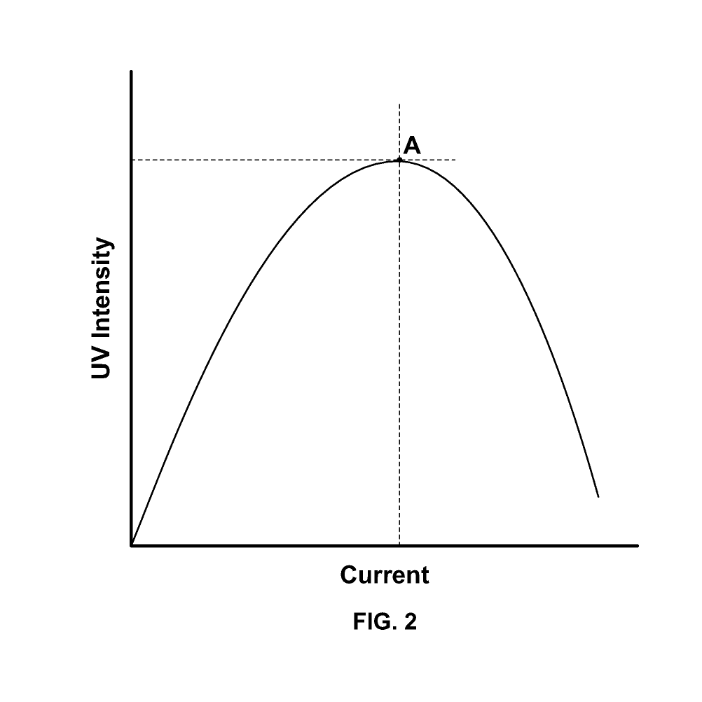

As discussed above, any temperature variation above or below the ideal operating temperature, i.e., the optimum lamp 102 cold-spot temperature, can decrease the effective UV output intensity of the lamp 102. With reference to FIG. 2, a chart illustrating a representative relationship between a supply of electrical current versus a UV lamp 102 output intensity is provided. It is generally desired to maintain the lamp 102 at the highest possible UV intensity, i.e., at point A, for the most effective implementation of the lamp 102 for purification purposes. Unlike typical lamps which become brighter with greater electrical current being supplied, UV lamps 102 generally reach a peak of UV output intensity, e.g., point A, as the electrical current supplied is increased and drop below the peak UV output intensity if the electrical current supplied continues to increase. The UV intensity at point A can thereby be maintained if the electrical current supplied corresponds to the value at point A. It should be understood that when the electrical current and the UV intensity are maintained at point A, the optimum lamp 102 cold-spot temperature can also be maintained.

It should further be understood that the representative chart of FIG. 2 represents the relationship between electrical current and UV output intensity for one temperature, e.g., one environment operating temperature, one current cold-spot temperature of the lamp 102, and the like. For example, if the optimum lamp 102 cold-spot temperature is approximately 117° F., for a specific environment operating temperature, the electrical current input must be maintained at point A to maintain the lamp 102 at the optimum cold-spot temperature. The representative chart of FIG. 2 can be varied for alternative temperatures, e.g., alternative environment operating temperatures or cold-spot temperatures. Thus, if the environment operating temperature drops, another representative chart or algorithm based on correlation data representative of the relationship between electrical current, environment operating temperatures, and UV output intensity can be programmed into the processor 108 to indicate the electrical current which would be required to maintain the UV output intensity at the peak, i.e., at point A. Thus, for each varying environment operating temperature and/or cold-spot temperature measured with the temperature sensor 104, the processor 108 can include programmed therein a plurality of algorithms and relationships indicating the optimum electrical current input required to maintain the lamp 102 at the optimum cold-spot temperature, thereby dynamically maintaining the optimum UV output intensity.

With respect to embodiments of the system 100 dependent on a measurement of the cold-spot temperature with the temperature sensor 104, the temperature sensor 104 can be installed internally or externally of the lamp 102 to measure the actual cold-spot temperature of the lamp 102 during use. As an example, the optimum cold-spot temperature for a given lamp may be approximately 117° F. The exemplary ballast 106 can be designed to provide a range of lamp 102 currents from a minimum to a maximum to maintain the lamp 102 at an optimum cold-spot temperature when the actual cold-spot temperature of the lamp 102 varies.

When a lamp 102 being dependent on a measured cold-spot temperature is turned on, it can start-up in the following exemplary methods. In one exemplary start-up method, the lamp 102 can be started with the ballast 106 at a maximum operating current. Thus, the processor 108 can be programmed to initially regulate the ballast 106 to supply a maximum operating current to the lamp 102. During the first several minutes of operation, the lamp 102 temperature, i.e., the lamp 102 cold-spot temperature, can gradually increase. When the optimum operating cold-spot temperature of, e.g., approximately 117° F., has been reached, the processor 108 can be programmed to initiate a reduction of the current being supplied by the ballast 106 to the lamp 102. In particular, the reduction of current being supplied to the lamp 102 can be continued until the cold-spot temperature of the lamp 102 has been stabilized at the optimum operating cold-spot temperature, e.g., approximately 117° F. As discussed above, it should be understood that the optimum cold-spot operating temperature can vary depending on the type of lamp 102 being implemented. Thus, the processor 108 for each specific lamp 102 can include programming therein to regulate the supply of current for the appropriate optimum operating cold-spot temperature.

In another exemplary start-up method, the lamp 102 can be started with the ballast 106 at a minimum operating current. Thus, the processor 108 can be programmed to initially regulate the ballast 106 to supply a minimum operating current to the lamp 102. During the first several minutes of operation, the lamp 102 temperature, i.e., the lamp 102 cold-spot temperature, can increase and stabilize below the optimum operating cold-spot temperature of, e.g., approximately 117° F. When the lamp 102 cold-spot temperature has stabilized, the processor 108 can be programmed to initiate a gradual increase of the current being supplied by the ballast 106 to the lamp 102. In particular, the gradual increase of current being supplied to the lamp 102 can be continued until the optimum operating cold-spot temperature of, e.g., approximately 117° F., has been reached. Once the optimum operating cold-spot temperature has been reached, the processor 108 can be programmed to maintain the supply of current necessary for maintaining the lamp 102 at the optimum operating cold-spot temperature based on the algorithms or relationships programmed therein. In some embodiments, rather than starting the ballast 106 at a maximum or a minimum operating current, the ballast 106 can be started at an intermediate predetermined operating current.

As discussed above, in some embodiments, the temperature sensor 104 can be placed in the operating environment surrounding the lamp 102, e.g., in the media or fluid within the internal space 114 of the housing 112. For example, in a swimming pool water purification system, the temperature sensor 104 senses the temperature of the swimming pool water that enters the housing 112 through the inlet 116 and into the internal space 114 surrounding the lamp 102. In such embodiments, the influence of the operating environment temperature (e.g., the swimming pool water temperature) on the lamp 102 temperature much be determined for a given lamp 102 model type. In particular, as discussed above, algorithms including correlation data can be developed for a specific lamp 102 type based on the relationship between the variety of operating environment temperatures and the current which needs to be supplied at each operating environment temperature to maintain the lamp 102 at the optimum operating cold-spot temperature. The algorithms can be programmed into the processor 108 and can provide an ideal lamp current for a given water temperature.

For example, if an optimum cold-spot temperature of a lamp 102 is approximately 117° F., the programmed algorithms and/or correlation data can show that at a water temperature of approximately 50° F., a current of approximately 800 mA would create and maintain the lamp 102 at the optimum cold-spot temperature. However, if the water temperature increased to approximately 80° F., the algorithms and/or correlation data can indicate that the lamp 102 current would need to be reduced to approximately 500 mA to maintain the lamp 102 at the optimum cold-spot temperature. Thus, the programmed algorithms and/or correlation data for the relationship between the operating environment temperatures and the current being supplied to the lamp 102 to maintain the lamp 102 at an optimum cold-spot temperature can be programmed into the processor 108 to dynamically regulate the ballast 106 such that the lamp 102 can be maintained at the optimum cold-spot temperature when the operating environment temperature changes over time.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

1. A lamp ballast system, comprising:
a lamp,
at least one temperature sensor positioned proximate to the lamp or incorporated into the lamp,
a ballast providing an electrical current to the lamp, and
a processor,
wherein the processor receives a sensed temperature from the at least one temperature sensor and, in response to the sensed temperature, directs a control signal to the ballast to regulate the electrical current provided to the lamp to maintain the lamp at an optimal operating temperature.

2. The system according to claim 1, wherein the lamp is an ultraviolet lamp.

3. The system according to claim 1, comprising a housing including an inlet and an outlet for introducing fluid to be purified into the housing and for discharging purified fluid from the housing, wherein the lamp is positioned within the housing.

4. The system according to claim 3, wherein the at least one temperature sensor is positioned inside the housing at the inlet, at the outlet, or at both the inlet and the outlet.

5. The system according to claim 1, wherein the at least one temperature sensor is at least one of a thermocouple, a thermistor, and a microchip.

6. The system according to claim 1, wherein the processor comprises a database therein configured to be programmed with at least one algorithm, the at least one algorithm representing a relationship between the electrical current required to maintain the lamp at the optimal operating temperature and a variety of sensed temperatures.

7. The system according to claim 1, wherein the sensed temperature is at least one of (i) an environment operating temperature and (ii) a lamp cold-spot temperature.

8. The system according to claim 1, wherein the optimal operating temperature is an optimal lamp cold-spot temperature.

9. The system according to claim 8, wherein the optimal lamp cold-spot temperature generates an optimal ultraviolet output intensity from the lamp.

10. A method of maintaining a lamp at an optimal operating temperature, the method comprising:
providing a lamp ballast system, the lamp ballast system including (i) the lamp, (ii) at least one temperature sensor positioned proximate to the lamp or incorporated into the lamp, (iii) a ballast providing an electrical current to the lamp, and (iv) a processor,
receiving a sensed temperature, via the processor, from the at least one temperature sensor, and
in response to the sensed temperature, directing a control signal, via the processor, to the ballast to regulate the electrical current provided to the lamp to maintain the lamp at the optimal operating temperature.

11. The method according to claim 10, wherein the lamp ballast system comprises a housing including an inlet and an outlet for introducing fluid to be purified into the housing and for discharging purified fluid from the housing, and wherein the lamp is positioned within the housing.

12. The method according to claim 11, comprising positioning the at least one temperature sensor inside the housing at the inlet, at the outlet, or at both the inlet and the outlet.

13. The method according to claim 12, comprising sensing the sensed temperature at one of (i) an operating environment surrounding the lamp or (ii) a lamp cold-spot.

14. The method according to claim 10, wherein maintaining the lamp at the optimal operating temperature comprises generating an optimal ultraviolet output intensity from the lamp.

15. The method according to claim 10, comprising starting the lamp by providing a maximum operating current to the lamp.

16. The method according to claim 15, comprising directing a reduction control signal to the ballast to reduce the electrical current provided to the lamp when the optimal operating temperature is reached.

17. The method according to claim 10, comprising starting the lamp by providing a minimum operating current to the lamp.

18. The method according to claim 17, comprising directing an increase control signal to the ballast to increase the electrical current provided to the lamp to reach the optimal operating temperature.

19. The method according to claim 10, comprising programming at least one algorithm into a database of the processor, the at least one algorithm representing a relationship between the electrical current required to maintain the lamp at the optimal operating temperature and a variety of sensed temperatures.

20. A non-transitory computer readable storage medium storing instructions, wherein execution of the instructions by a processor causes the processor to implement a method of maintaining a lamp at an optimal operating temperature, comprising:
receiving a sensed temperature, via the processor, from at least one temperature sensor of a lamp ballast system, the lamp ballast system including (i) the lamp, (ii) the at least one temperature sensor positioned proximate to the lamp or incorporated into the lamp, (iii) a ballast providing an electrical current to the lamp, and (iv) the processor, and
in response to the sensed temperature, directing a control signal, via the processor, to the ballast to regulate the electrical current provided to the lamp to maintain the lamp at the optimal operating temperature.
